(19) United States
(12) Patent Application Publication
(10) Pub. No.: US 2004/0199199 A1
(43) Pub. Date: Oct. 7, 2004

(54) Filter and method of making a filter

(75) Inventors: Jeff Krolik, Campbell, CA (US); Hoa Vinh Vo, Campbell, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/405,547

(22) Filed: Apr. 2, 2003

(51) Int. Cl. A61M 29/00

(52) U.S. Cl. 606/200

(57) ABSTRACT

An embolic protection filtering device and method of making the same. In at least some embodiments, a method of making an embolic protection filter includes providing a mandrel and a filter material, advancing the mandrel toward the filter material and stretching the filter material, and drilling a plurality of holes in the filter material.

### FIELD OF THE INVENTION

The present invention pertains to embolic protection. More particularly, the present invention pertains to embolic protection filters and methods of making the same.

### BACKGROUND

Heart and vascular disease are majors problem in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

### BRIEF SUMMARY

The present invention pertains to an embolic protection filter device and devices and method for manufacturing the same. An embolic protection device may include a filter coupled to an elongate shaft or guidewire. The filter can be generally configured to be disposed in a body lumen such as a blood vessel and filter out debris.

In at least some embodiments, a method of manufacturing an embolic protection filter device includes providing an embolic protection filter manufacturing assembly, a mandrel, a stretch frame, and a filter material. The mandrel may then be advanced toward the filter material and stretch a portion thereof. The filter material may also be subjected to additional manufacturing steps including hole drilling and annealing.

### BRIEF DESCRIPTION OF THE DRAWINGS

FIG. 1 is a plan overview of an example embolic protection filter device;

FIG. 2 is a side view of an example embolic protection filter manufacturing assembly;

FIG. 3 is a perspective view of a partially stretched filter material;

FIG. 4 is a side view of the stretch frame and the filter material;

FIG. 5 is an exploded view of some components of the hole drilling assembly; and

FIG. 6 is a side view of a filter frame and filter material, wherein a plurality of holes are formed within the filter material; and

FIG. 7 is enlarged view of the holes within the filter material.

### DETAILED DESCRIPTION

The following description should be read with reference to the drawings wherein like reference numerals indicate like elements throughout the several views. The detailed description and drawings illustrate example embodiments of the claimed invention.

Embolic protection devices and, more particularly, embolic protection filters may be manufactured by a number of different methods. For example, a method of dip polymeric molding where a mandrel may be dipped into a container of liquid polymeric filter material and then the filter material may be allowed to solidify. Once solidified, a plurality of holes can be drilled into the filter material and the new “filter” can be attached to a guidewire. Although this manufacturing method is useful, there is an ongoing need for new and improved embolic protection devices and methods of manufacturing embolic protection devices.

The present invention includes a number of example embolic protection devices and methods of manufacturing embolic protection filters. In at least some embodiments, the method of manufacturing includes providing a generally planar filter material and stretching the filter material with a mandrel. The filter material can then be further processed (e.g., drilled, annealed, coupled to a filter frame, attached to a guidewire, etc.) and used as part of an embolic protection device. This method may incorporate a number of desirable characteristics. For example, this method may enhance the consistency of filter thickness (relative to dip molding or other methods), allow for a greater variety of materials that can be used for the filter material or for other parts of the device, reduce manufacturing costs by incorporating more common components and less specialized (e.g. heat resistant) equipment, improved strength and/or performance, etc. Moreover, it is believed that including an annealing step after drilling holes in the filter material increases the strength of the formed and drilled filter material, even when the holes are enlarged. Other features, elements, and properties of the invention are described in more detail below.

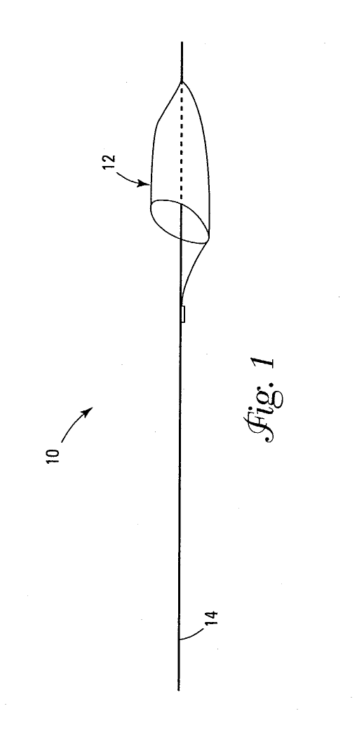

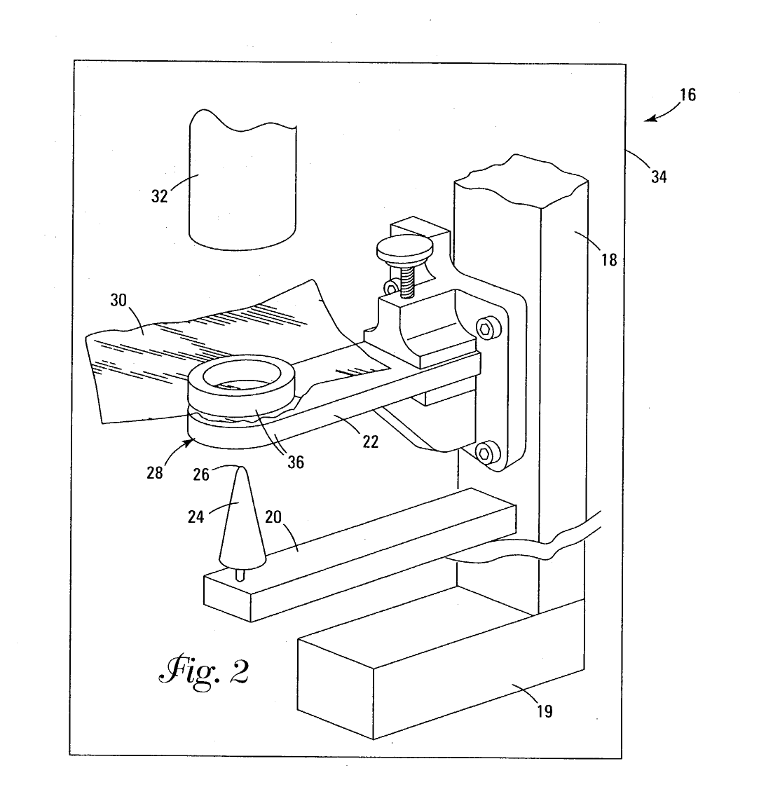

FIG. 1 is a plan overview of an example embolic protection filter device 10. Device 10 includes an embolic protection filter 12 coupled to an elongate shaft or guidewire 14. Filter 12 may be manufactured according to the dip molding protocol discussed above or, alternatively, filter 12 may be manufactured by other methods including those described in more detail below. For example, FIGS. 2-4 illustrate example device intermediates and manufacturing steps appropriate for manufacturing device 10.

FIG. 2 is a side view of an example embolic protection filter manufacturing assembly 16 that can be used to manufacture device 10. Assembly 16 includes a shaft 18 extending from a base member 19. Shaft 18 may include a first arm 20 and a second arm 22 extending therefrom. First arm 20 may include a forming mandrel 24 having a generally tapered distal end 26 coupled thereto. Second arm 22 may include a filter hoop assembly or holding member 28 adapted and configured for holding a filter material 30. A heat source 32 may also be included and be positioned adjacent filter material 30, for example above filter material 30 and coupled to shaft 18 by an arm. Assembly 16 may be contained within a chamber 34, for example, to allow for temperature and pressure control. One or more temperature and pressure control conduits (not shown) may be connected to chamber 34 so that the temperature and pressure within chamber 34 can be controlled.

At least some of the components listed above may be similar to other typical laboratory devices known to those of ordinary skill in the art. For example, shaft 18 and base member 19 may comprise a ring stand or other related device commonly used in a laboratory setting. Additionally, first arm 20 and second arm 22 may be similar to other arm or clamping devices that are, for example, used with ring stands. In at least some embodiments, first arm 20 and/or second arm 22 are slidably and/or detachably connectable to shaft 18.

A number of preliminary set-up steps may be carried out prior to or concurrently with forming filter 12. For example, chamber 34 may be pre-heated to a temperature of about 300-400° F. (for example, about 352° F.±5° F.). Heat source 32 may also be turned on and configured to operate with a desired setpoint temperature in the range of about 200-300° F. (for example, about 240° F.±5° F.). The above warm-up steps may extend over a period of time, for example about 15 minutes or longer. In addition, the position and configuration of first arm 20 and second arm 22 may also be set. For example, first arm 20 and second arm 22 may be set so that distal end 26 of mandrel is positioned about 100 to 200 mm (for example, about 144 mm±2 mm) away from holding member 28. Additionally, the heat source 32 may be disposed about 15-35 mm (for example, about 25 mm±3 mm) away from holding member 28.

In at least some embodiments, assembly 16 may be configured so that first arm 20 is located below second arm 22, and so that forming mandrel 24 is disposed below filter material 30 as shown in FIG. 2. However, it can be appreciated that the exact location of each of the above components may be varied without departing from the spirit of the invention. For example, first arm 20 may be located above second arm 22. Alternatively, the above components may be arranged horizontally.

Filter material 30 is generally configured by disposing at least a portion thereof adjacent holding member 28. For example, holding member 28 may include one or more rings 36 and filter material 30 may be disposed between rings 36. In some embodiments, one of the rings 36 may be coupled to or integral with arm 22. Rings 36 may comprise a number of different configurations or forms. For example, rings 36 may be configured to be threadably joined, joined by friction fit, be arranged adjacent one another, overlap in part with one another, etc. Filter material 30 may be positioned to that it encompasses the central holes or channels of rings 36.

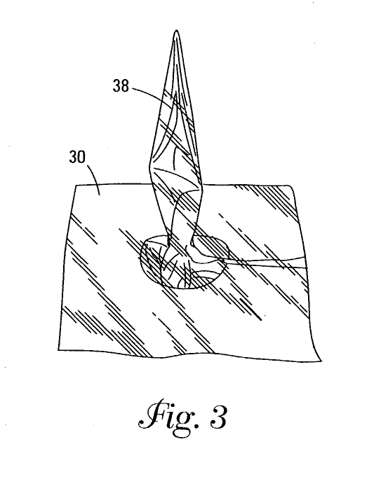

Mandrel 24 may be used to form filter 12 by advancing first arm 20 toward filter material 30 so that mandrel 24 contacts and stretches filter material 30. This can occur, for example, by sliding arm 20 along shaft 18 toward filter material 30 or by sliding second arm 22 (and holding member 28) toward mandrel 24. Ultimately, distal end 26 of mandrel 24 will contact filter material 30 (for example, adjacent the portion of filter material 30 disposed at the central holes or channels of rings 36) and, as either arm 20/22 is further advanced, begin to stretch filter material 30 and define a stretched portion 38 of filter material 30 that is best seen in FIG. 3. Stretched portion 38 may be used with additional manufacturing steps to form filter 12.

In at least some embodiments, when mandrel 24 contacts and stretches filter material 30, stretched portion 38 generally conforms to the shape of mandrel 24 (i.e., tapered distal end 26) and is disposed over mandrel 24. According to this embodiment, the shape of distal end 26 is generally similar or a precursor to the desired shape of filter 12. The desired shape may be generally tapered, cone-shaped, narrowed, or the like. Thus, the shape of mandrel 24 may at least in part be configured to alter the generally planar shape of filter material 30 toward the final shape of filter 12. It can be appreciated that different embodiments of mandrel 24 may have different shapes and can be used to form differently shaped filters 12 without departing from the spirit of the invention.

At least a portion of forming mandrel 24 (for example, distal end 26) may be comprised of or coated with a generally lubricious material such as polytetrafluoroethylene (PTFE). This may, for example, allow stretched portion 38 to be more easily separated from mandrel 24. The remaining portions of mandrel 24 may be comprised of essentially any appropriate material such as a metal, metal alloy, polymer, metal-polymer composite, and the like.

As suggested above, filter material 30 may comprise a generally planar sheet or film of material. In at least some embodiments, filter material 30 is polymeric. Some examples of suitable polymers include, but should not be limited to, fluorinated ethylene propylene (FEP), polymer, polyethylene (PE), polypropylene (PP), polyvinylchloride (PVC), polyurethane, polytetrafluoroethylene (PTFE), polyether block amide (PEBA), polyether-ether ketone (PEEK), polyimide, polyamide, polyphenylene sulfide (PPS), polyphenylene oxide (PPO), polysufone, nylon, perfluoro(propyl vinyl ether) (PFA), polyurethane polycarbonate copolymer (for example, BIONATE®), combinations thereof, and the like.

In at least some embodiments, a plurality of sheets of filter material 30 may be used. The sheets may be comprised of the same materials or, alternatively, may be comprised of differing materials. For example, some of the sheets of filter material 30 may be comprised of materials that are generally softer, stretchy, stronger, harder, more scratch resistant, etc. Additionally, one or more of the sheets of filter material 30 may include a drug or medicament. Some examples of suitable medicaments may include anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin, prostaglandin inhibitors, platelet inhibitors and tick antiplatelet peptides; vascular cell growth promotors such as growth factor inhibitors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; and cholesterol-lowering agents; vasodilating agents; agents which interfere with endogenous vascoactive mechanisms; anti-sense DNA and RNA; DNA coding for (and the corresponding proteins) anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin like growth factor, cell cycle inhibitors including CD inhibitors, thymidine kinase (“TK”) and other agents useful for interfering with cell proliferation, and the family of bone morphogenic proteins (“BMP's”) including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, BMP-16, “hedgehog” proteins; or other appropriate substances.

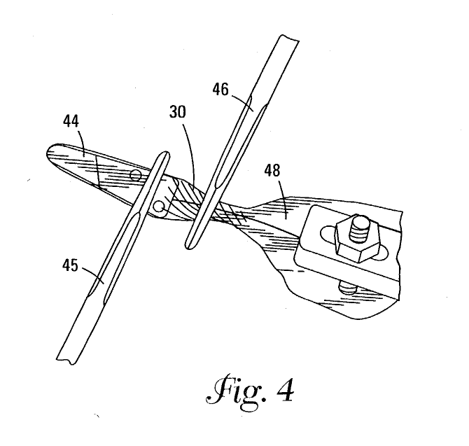

Once stretched and separated from mandrel 24, filter material 30 may be subjected to further manufacturing steps. For example, filter material 30 may be disposed over a stretch frame 44 as illustrated in FIG. 4. Stretch frame 44 may comprise a generally planar frame that may serve as a template for drilling holes in filter material 30 as described in more detail below. As shown in FIG. 4, stretch frame 44 and filter material 30 may be held in place with a suitable clamping device 45 and may be heat sealed, for example with a pre-heated smooth jawed hemostat or other suitable clamping device 46. Additionally, the excess portion 48 of filter material 30 may be cut off. For example, excess portion 48 may be twisted a number of times and then cut off adjacent stretch frame 44.

The thickness of the remaining portion of filter material 30 (i.e., the portion disposed at stretch frame 44) may be measured by a suitable measuring device technique such as beta back scattering. In addition to determining the thickness of filter material 30, measuring allows a technician to determine if any portions of filter material 30 have a thickness that is too thin or too thick. In some embodiments, the thickness of filter material 30 is in the range of about 0.00005 to about 0.002 inches. Measuring may also allow the technician to detect any rips or tears within filter material 30.

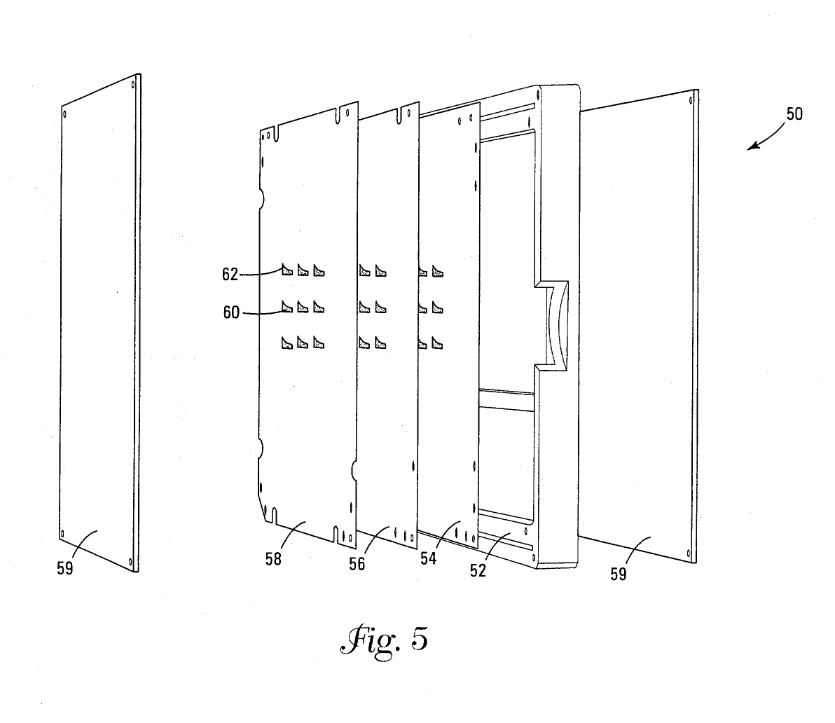

A plurality of holes may be formed in filter material 30. A number of methods may be used to form the holes. For example, FIG. 5 illustrates some components of a suitable hole drilling assembly 50. Assembly 50 is compatible for use with a hole drilling device, for example a laser drilling device. In at least some embodiments, assembly 50 includes a frame 52, a base layer 54, a position layer 56, a mask 58, and may include one or more end covers 59. Each of layers 54/56/58 may include a plurality of holes 60. Frame 52 is generally configured for holding the other layers and be positioned adjacent the hole drilling device. Base layer 54 can be positioned on top of frame 52. Position layer 56 can be positioned on top of base layer 54 and includes holes 60 that are each adapted and configured for holding stretch frame 44. It can be seen in FIG. 5 that holes 60 have a shaped that is similar to stretch frame 44 with an additional enlarged region 62 that permits a technician to place or remove stretch frame 44 from hole 60, for example with a forceps or other suitable device. Mask 58 can be positioned on top of position layer 56.

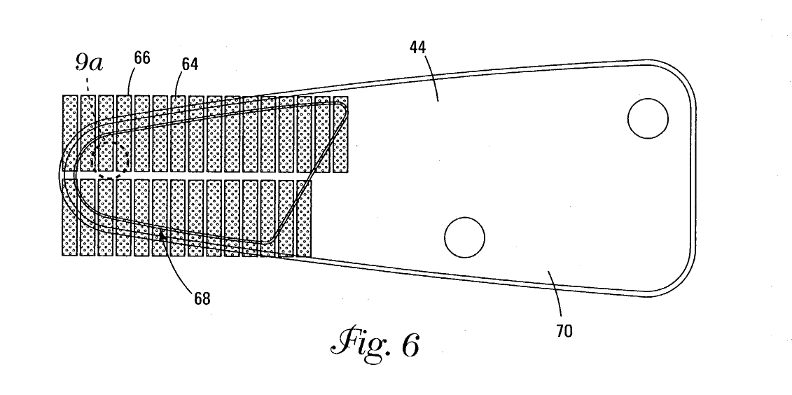

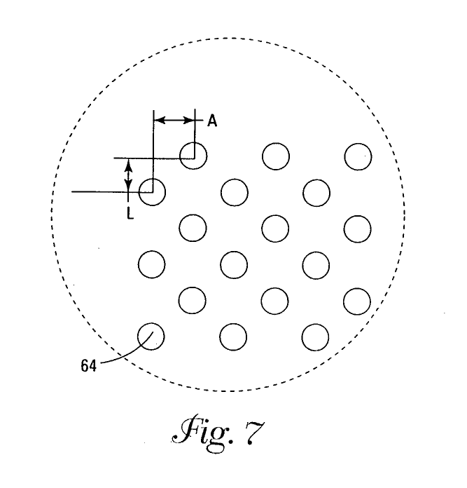

Position layer 56 can be loaded with a plurality of stretch frames 44 (each having filter material 30 disposed thereon) and hole drilling assembly 50 may be positioned adjacent the drilling device. Because stretch frames 44 may be generally planar, filter material 30 on stretch frames 44 may be generally flat. This may be desirable, for example, by allowing the laser drilling device to be set to a singular laser focal length, which may increase the efficiency, accuracy, and consistency of drilling. The drilling device can drill a plurality of holes 64 within filter material 30 as generally shown in FIG. 6 and enlarged in FIG. 7. In some embodiments, the drilling device may be coupled to a computer system that is programmed to drill holes according to a series of repeat patterns 66. The exact dimensions of repeat pattern 66 can be altered for different embodiments. For example, repeat pattern 66 may be configured to result in holes 64 being spaced longitudinally (dimension L) about 90-150 μM (e.g., about 109 μM) and axially (dimension A) about 100-150 μM (e.g., about 127 μM). Additionally, repeat pattern 66 may also define the size of holes 64. For example, holes 66 may have diameter in the range of about 60-100 μM (e.g., about 80 μM).

FIG. 6 also includes an enlarged illustration of stretch frame 44. From this illustration, it can be seen that only a portion of filter material 30 disposed adjacent stretch frame 44 will ultimately be included in filter 12. For example, as seen in FIG. 6, stretch frame may include a filter region 68 and a handling region 70. Filter region 68 corresponds with essentially the portion of filter material 30 that will be included with filter 12. Handling region 70 can be used to hold, move, or otherwise manipulate stretch frame 44. Inclusion of handling region 70 allows the technician to be able to manipulate stretch frame 44 without coming into contact with filter material 30 (at filter region 68).

Filter material 30 (either while still disposed adjacent stretch frame 44 or separated therefrom) may also annealed. It is believed that annealing increases the size of holes 64 without altering the strength of filter material 30 (and/or filter 12). Thus, annealing allows holes 64 to be drilled with a size that is smaller than what is desired for filter 12 (which increases the strength of drilled filter material 30 relative to one with larger holes) and then annealed so that holes 64 enlarge (to the desired size) without sacrificing any strength characteristics. It can be appreciated that the annealing conditions can be adapted to result in the desired alteration in size of hole 64. For example, filter material 30 may be placed in an 85° oven for about 1 minute and then allowed to cool. Holes 64 can be measured for size and compared with the size and pattern defined by repeat pattern 66.

In some embodiments, filter material 30 may be separated from stretch frame 44 after annealing. At this or at essentially any appropriate time, filter material 30 may then be additionally processed. For example, filter material 30 may be coupled to a filter frame. The filter frame may provide additional structural support to filter 12. In some embodiments, the filter frame may be comprised a shape-memory alloy, for example nickel-titanium alloy. This type of filter frame may allow filter 12 to shift between an expanded and a collapsed configuration. The filter frame and/or filter 12 may be coupled to shaft 14.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A method of manufacturing an embolic protection filter, comprising the steps of:
providing a mandrel;
providing a filter material;
bringing the mandrel and the filter material into contact such that the filter material stretches and so that at least a portion of the filter material generally conforms to the shape of the mandrel; and
forming a plurality of holes in the filter material.

2. The method of claim 1, wherein the step of bringing the mandrel and the filter material into contact includes heating the filter material with a heat source.

3. The method of claim 1, wherein the step of bringing the mandrel and the filter material into contact further includes heat sealing a portion of filter material.

4. The method of claim 1, wherein the step of forming a plurality of holes in the filter material includes laser drilling holes in the filter material.

5. The method of claim 1, further comprising the step coupling the filter material to an elongate guidewire.

6 The method of claim 1, further comprising the step of measuring the thickness of the filter material adjacent the stretched portion thereof that generally conforms to the shape of the mandrel.

7. The method of claim 1, further comprising the step of annealing the filter material.

8. A method of manufacturing an embolic protection filter, comprising the steps of:
providing an embolic protection filter manufacturing assembly, the assembly including a base member, a shaft extending from the base member, a first arm coupled to the shaft and including a mandrel member, and a second arm coupled to the shaft and including a filter material holding member;
coupling a filter material to the filter material holding member;
bringing the mandrel member into contact with the filter material such that the filter material stretches to define a stretched filter-shaped member;
disassociating the mandrel member from the filter-shaped member;
coupling the filter-shaped member to a stretch frame;
disposing the filter-shaped member and stretch frame adjacent a drilling apparatus;
drilling a plurality of holes in the filter-shaped member with the drilling apparatus; and
annealing the filter-shaped member.

9. The method of claim 8, wherein the filter material holding member includes a filter hoop having an opening and wherein the step of coupling a filter material to the filter material holding member includes disposing the filter material adjacent the filter hoop so that at least a portion of the filter material is disposed within the opening.

10. The method of claim 8, further comprising the step of heating the filter material.

11. The method of claim 8, further comprising the step of heat sealing a portion of filter material.

12. The method of claim 8, wherein the drilling apparatus includes a laser drilling apparatus, and wherein the step of drilling a plurality of holes in the filter-shaped member with the drilling apparatus includes laser drilling holes in the filter material.

13. The method of claim 8, wherein the step of annealing the filter-shaped member enlarges the holes.

14. The method of claim 8, further comprising the step coupling the filter-shaped member to an elongate guidewire.

15. The method of claim 8, further comprising the step of measuring the thickness of the filter-shaped member.

16. An embolic protection filter assembly, comprising:
an embolic protection filter forming assembly, the assembly including a moveable mandrel member and a filter material holding member;
a filter material coupled to the filter material holding member; and
means for drilling one or more holes in the filter material.

17. An embolic protection filter assembly, comprising:
an embolic protection filter forming assembly, the assembly including a mandrel, a stretch frame having a proximal end and a tapered distal end, and a filter material holding member;
a filter material coupled to the filter material holding member;
a support member for moving the mandrel into contact with the filter material;
wherein contact between the mandrel and the filter material results in stretching of the filter material and at least a portion of the filter material conforming to the shape of the mandrel; and
a drilling apparatus for drilling a plurality of holes into the filter material.
